(19) United States
(12) Patent Application Publication
(10) Pub. No.: US 2024/0189187 A1
(43) Pub. Date: Jun. 13, 2024

(54) FLUID TRANSFER DEVICE AND CLOSED MEDICINE TRANSFER SYSTEM

(71) Applicant: B. Braun Melsungen AG, Melsungen (DE)

(72) Inventors: Florin Kopp, Schortens (DE); Karl-Martin Berg, Melsungen (DE); Konstantin Krug-Sauer, Gudensberg (DE); Johannes Bolz, Kassel (DE)

(21) Appl. No.: 18/284,954

(22) PCT Filed: Mar. 28, 2022
(86) PCT No.: PCT/EP2022/058136

§ 371 Date: Sep. 29, 2023

(30) Foreign Application Priority Data
Mar. 31, 2021 (DE) 10 2021 108 157.2

(51) Int. Cl. A61J 1/20; A61J 1/14

(52) U.S. Cl. CPC A61J 1/2051; A61J 1/1406; A61J 1/201; A61J 1/2055; A61J 1/2065; A61J 1/2096

(57) ABSTRACT

A fluid transfer device for use in medical/pharmacological applications a method of use, and a closed medicine transfer system. The fluid transfer device includes a first adapter unit having a first sealing element and a second adapter unit having a second sealing element. The first adapter unit and second adapter unit, when connected, provide a fluid connection between the first adapter unit and the second adapter unit and prevent, when disconnected, the transfer of fluid out of the first adapter unit and the second adapter unit. A fluid connection between the first adapter unit and second adapter unit is established by a combined pressing movement towards one another and a rotational movement with respect to one another, only after a constant pressure has been set which the first sealing element and the second sealing element apply against each other.

### CROSS-REFERENCE TO RELATED APPLICATIONS

This application is the United States national stage entry of International Application No. PCT/EP2022/058136, filed Mar. 28, 2022, and claims priority to German Application No. 10 2021 108 157.2, filed Mar. 31, 2021. The contents of International Application No. PCT/EP2022/058136 and German Application No. 10 2021 108 157.2 are incorporated by reference herein in their entireties.

### FIELD

The present disclosure relates to a fluid transfer device for use in medical and/or pharmacological applications, comprising a first adapter unit comprising a first sealing element and a second adapter unit comprising a second sealing element, wherein the first adapter unit and the second adapter unit are configured to provide a fluid connection between the first adapter unit and the second adapter unit in a connected state and to stop or prevent the fluid transfer out of the first adapter unit and the second adapter unit in an unconnected or separated state.

Furthermore, the present disclosure relates to a use of such a fluid transfer device for transferring or conveying medicinal products, in particular CMR medicinal products (medicinal products which are carcinogenic/cancer-producing, mutagenic and reprotoxic/teratogenic), from a first container to a second container.

Finally, the present disclosure relates to a closed medicine transfer system comprising such a fluid transfer device and at least a first container, wherein the fluid transfer device is configured to provide a fluid connection between the first container and a second container or between the first container and a patient.

### BACKGROUND

Closed (medicinal product) transfer systems (also called ‘Closed System Transfer Devices—CSTD’) are known from the prior art and are typically used when transferring hazardous or harmful medicinal products from one container to another container or from a container to a patient. For example, closed medicine transfer systems are used to transfer CMR medicinal products, i.e. carcinogenic, mutagenic and reprotoxic medicinal products, from an ampule to a syringe or from the syringe to an infusion bag or from the infusion bag to a patient. In other words, closed medicine transfer systems are used both in the preparation/production/manufacturing of ready-to-use solutions and in the administration of the ready-to-use solutions.

CMR medicinal products or CMR drugs are used, for example, in cancer therapy and combat particularly growth-intensive tumor cells in therapeutic applications. As already indicated, these medicinal products have carcinogenic, mutagenic or reproductive toxicity properties due to their mechanism of action. Against this background, it is essential to prevent medical personnel who are not treated with CMR medicinal products from being exposed to these dangerous or harmful medicinal products or coming into direct/immediate contact with them during the preparation or administration of CMR medicinal products.

Closed medicine transfer systems should therefore, in a known manner, prevent the transfer of impurities/contaminants from the environment into the closed system and the escape of dangerous/harmful drug or vapor concentrations from the closed system, in particular mechanically.

An important component of a closed medicine transfer system is a fluid transfer device/a port system/coupling system, which consists of two (adapter) units/components/parts/coupling partners, which can be connected to each other to enable the transfer of the CMR medicinal product via the fluid transfer device, and which can be separated from each other and in the separated state prevent/stop a fluid transfer out of the two units. The coupling/connection/contact points of the two units should be as dry as possible after separation, i.e. the CMR medicinal product should not leak or drip from the surfaces of the coupling partners in order to protect the environment from contamination.

With this background, coupling systems/fluid transfer devices, which have self-sealing properties and allow dry connection and separation of the coupling partners to the greatest possible extent, have become increasingly important in the preparation and administration of ready-to-use CMR medicinal products in recent years.

Closed medicine transfer systems with fluid transfer device(s), which in a connected state provide a fluid connection between a first (adapter) unit and a second (adapter) unit and in an unconnected or separated state prevent/stop the fluid transfer out of the first (adapter) unit and the second (adapter) unit are known, for example, from WO 2016/051390 A1, WO 2016/199133 A1, WO 2016/147178 A1, WO 2017/109776 A1, US2016/058667 A1, EP 2 139 442 B1, EP 2 606 872 B1, U.S. Pat. No. 8,511,352 B2, US 2018/0200498 A1, EP 2 753 396 B1 and U.S. Pat. No. 9,345,641 B2.

Known fluid transfer devices have substantially in common that they are connected by a translatory pressing movement of the two (adapter) units towards each other/by an opposite application of force on the two (adapter) units in the axial direction according to the plug-and-socket principle. When one (adapter) unit is plugged into the other (adapter) unit, a connected state of the two units is directly established, in which a fluid connection is provided between the first unit and the second unit.

In the case of known fluid transfer devices, it has been found that small amounts of moisture often remain on coupling surfaces, for example on elastic sealing elements of the two units, which are in contact with each other in the connected state.

Other disadvantages of known fluid transfer devices are complex handling, low flow rate, relatively large size, and poor accessibility and thus disinfectability of the coupling surfaces.

### SUMMARY

Against this background, the object of the present disclosure is to avoid or at least reduce the aforementioned disadvantages of the prior art. In particular, a fluid transfer device is to be provided which can be used as part/component of a closed medicine transfer system, which in its connected state enables a fluid transfer through it or provides a fluid connection through it, which in its separated/unconnected state prevents or stops a fluid transfer out of the coupling partners/adapter units, and which in particular achieves that the coupling surfaces of the coupling partners/adapter units are dry in the separated state, i.e. that no or hardly any moisture residues remain on them. In addition, the fluid transfer device is intended to be easy to handle, to provide a suitable flow rate through it for the application, not to be too large in terms of its size, and to have easily accessible and thus disinfectable coupling surfaces.

This object is solved by a fluid transfer device for use in medical and/or pharmacological applications. Advantageous embodiments and further developments are explained below.

The present disclosure relates firstly to a fluid transfer device for use in medical and/or pharmacological applications, comprising a first adapter unit comprising a first sealing element and a second adapter unit comprising a second sealing element, wherein the first adapter unit and the second adapter unit are configured to provide a fluid connection between the first adapter unit and the second adapter unit in a connected state and to prevent/stop fluid transfer out of the first adapter unit and the second adapter unit in an unconnected or separated state, wherein the first adapter unit and the second adapter unit are formed to establish the fluid connection between the first adapter unit and the second adapter unit by a combined pressing movement (translationally) towards each other and rotational movement (relative) to each other only after adjusting a constant/defined/predetermined pressure, which the first sealing element and the second sealing element apply against each other.

According to the present disclosure, it is thus ensured that between the adapter units/coupling partners/components/units of the fluid transfer device, in particular between the first sealing element and the second sealing element, which are brought into contact at their respective (outer) coupling surfaces, there is initially a constant or defined or predetermined pressure which the two sealing elements apply against/to each other, and only then, i.e. when the desired pressure is present/applied, is the fluidically connected state of the adapter units established. Preferably, the first sealing element and the second sealing element are thus only completely penetrated by a needle, or an interface/intersection/contact point of the first sealing element and the second sealing element is only penetrated by the needle when the two sealing elements already apply the desired, constant pressure against each other.

Preferably, the first sealing element and the second sealing element also apply the defined/constant/predetermined pressure against each other when separating the two adapter units until the needle has been retracted again via the interface of the first sealing element and the second sealing element. The mechanical separation of the two adapter units therefore preferably only takes place when the needle has been retracted again.

Thus, according to the disclosure, the fluidically connected state and the separated/fluidically unconnected state of the fluid transfer device are only established when the constant/defined pressure is applied to the two sealing elements. This ensures that hardly any fluid residues/fluid drops remain on the coupling surfaces of the sealing elements. This enables a dry connection and dry separation according to the disclosure.

In an advantageous manner, the first adapter unit and the second adapter unit are thus configured to assume, in addition to the connected state and the unconnected or separated state, a transfer state in which the first adapter unit and the second adapter unit are attached to each other and the first sealing element and the second sealing element are in contact with each other and apply the constant or defined pressure against each other, and a fluid connection is not yet provided between the first adapter unit and the second adapter unit.

According to the present disclosure, the adapter units are thus preferably not brought directly from the unconnected/separated state into the fluidically connected state by being plugging into each other (according to the plug-and-socket principle), but the two adapter units additionally assume a defined third state, namely the transfer state. Thus, the functions ‘establishment of a fluid connection’ and ‘application of the defined, constant pressure to each other by the two sealing elements’ are functionally and temporally separated. In other words, in a first step, the constant pressure is applied by the two sealing elements against each other, and in a second step following in time, the fluid connection between the two adapter units is established, in particular by the needle.

According to a preferred embodiment of the present disclosure, the functional and temporal separation of the two aforementioned functions is achieved in particular in that the first adapter unit and the second adapter unit are configured to be transferred from the unconnected or separated state to the transfer state by pressing on each other and rotating relative to each other by a first predetermined angle, and to be transferred from the transfer state to the connected state by continued rotation by a second predetermined angle.

An advantageous aspect of the present disclosure is the fact that a combined pressing and rotation movement by a user, for example medical personnel, is required to establish the (fluidically) connected state of the fluid transfer device. Thus, according to the present disclosure, it is not sufficient to move the two adapter units translationally towards each other and to insert one of the adapter units into the other one of the adapter units according to the plug-and-socket principle. In addition to the translational movement, a rotational movement or turning movement is required, in particular if the two adapter units are already pressed against each other. Preferably, rotation about the first predetermined angle causes the two adapter units to change from the separated/unconnected state to the transfer state. Starting from the transfer state, rotation around the second predetermined angle is preferred in order to transfer the two adapter units from the transfer/move state to the connected state.

Preferably, the two adapter units can be transferred from the connected state back to the transfer state by a (reverse or continued) rotational movement around the second predetermined angle, and from the transfer state back to the unconnected/separated state by a continued rotational movement in the selected rotational direction around the first predetermined angle.

Preferably, the second adapter unit comprises a hollow needle forming a fluid passage, and the hollow needle is received in the second sealing element in the unconnected or separated state and the transfer state and penetrates an/the interface between the first sealing element and the second sealing element only when the first adapter unit and the second adapter unit are transferred from the transfer state to the connected state. Preferably, the hollow needle has a so-called pencil point tip. Further preferably, the hollow needle has a lateral opening at/near its tip. Advantageously, the hollow needle establishes a fluidic connection between the two adapter units only after it has penetrated the interface between the first sealing element and the second sealing element and also completely penetrated the first sealing element of the first adapter unit. An inner diameter of the hollow needle and a size of the lateral opening are preferably suitably determined for adjusting a flow rate through the fluid transfer device.

A preferred embodiment is characterized in that the second adapter unit comprises a substantially hollow-cylindrical/sleeve-shaped component and a substantially cylindrical component, wherein the cylindrical component is received in the hollow-cylindrical/sleeve-shaped component (in particular at a distal end of the second adapter unit), and wherein rotation/turning of the hollow-cylindrical/sleeve-shaped component causes translational movement of the hollow-cylindrical/sleeve-shaped component and the cylindrical component relative to each other.

The terms ‘proximal’ and ‘distal’ are defined herein for the purposes of the present application as follows: ‘proximal’ means ‘closer to a user (medical personnel)’ and ‘distal’ means ‘further away from a user (medical personnel)’, assuming that when the fluid transfer device is connected and separated, the second adapter unit is located closer to the user, i.e. proximal, and the first adapter unit is located further away from the user, i.e. distal.

In other words, the second adapter unit preferably comprises at least two members/components that can be rotated relative to each other, and a turning/twisting/rotation of the two members/components relative to each other preferably causes a translational movement of the two members/components relative to each other.

In yet other words, the second adapter unit is thus configured to convert a rotational movement applied to its hollow-cylindrical component by a user (medical personnel) into a translational movement of the hollow-cylindrical component and the cylindrical component relative to each other. That is, when the user keeps pressing the first adapter unit against the second adapter unit and turns the hollow-cylindrical component of the second adapter unit, the hollow-cylindrical component and the cylindrical component move/shift translationally/axially relative to each other.

Preferably, the cylindrical component moves/shifts in a proximal direction, i.e. further into the interior of the hollow-cylindrical component, or respectively the hollow-cylindrical component preferably moves in a distal direction, when the adapter unit is moved from the separated state into the transfer state and further from the transfer state into the connected state. If the adapter unit is moved from the connected state via the transfer state into the separated state, the situation is preferably reversed.

Advantageously, the cylindrical component has a pin-shaped, projection projecting radially outward, and the hollow-cylindrical/sleeve-shaped component has a motion link/motion link path formed like a thread/thread pitch, and the pin-shaped projection of the cylindrical component is received in the motion link of the hollow-cylindrical/sleeve-shaped component. The motion link is preferably formed as a groove-like recess in the hollow-cylindrical component.

The motion link/motion-link path preferably runs obliquely/has a specific inclination, i.e. preferably extends both in the circumferential direction and in the axial direction of the sleeve-shaped/hollow-cylindrical component.

Preferably, the pin-like projection of the cylindrical component in the unconnected/separated state of the two adapter units is located at a (distal) stop of the motion link/motion-link path, so that a turning of the hollow-cylindrical component relative to the cylindrical component is only possible in one direction.

Advantageously, the pin-like projection of the cylindrical component moves into/along the motion link/motion-link path when the hollow-cylindrical component is turned, causing the axial/translational displacement of the two components relative to each other.

When the connected state of the two adapter units is reached, the pin-like projection is preferably located in a portion of the motion link/motion-link path without inclination, i.e. in a portion that extends only in the circumferential direction. In other words, the motion link/motion-link path advantageously has at least one portion without inclination. When the pin-like projection is located in the portion of the motion link without inclination, there is advantageously no further translational displacement of the cylindrical component and the hollow-cylindrical component relative to each other and thus no further translational displacement of the hollow-cylindrical component relative to the first adapter unit. The user thus recognizes in an advantageous manner that the connected state of the two adapter units has been reached when he can no longer detect any relative translational displacement.

Preferably, the motion link/motion-link path comprises a first portion in which the motion link extends from a first distal stop in the circumferential direction and in the axial direction (distal-proximal) of the hollow-cylindrical component, a second portion which is the portion without inclination, and a third portion which extends from the second portion in the circumferential direction and in the axial direction (proximal-distal) of the hollow-cylindrical component to a second distal stop. Advantageously, the hollow-cylindrical component can be turned in any direction (clockwise/counterclockwise) starting from the portion without inclination/from the connected state of the two adapter units in order to transfer the adapter units from the connected state back via the transfer state to the separated state. Alternatively, the motion link may have only the first portion and the second portion (not the third portion).

Overall, the motion link/motion-link path thus preferably defines an axial/translational displacement range of the sleeve-shaped component with respect to the cylindrical component.

An advantageous embodiment is characterized in that: the first adapter unit has a proximal wall having a first through-hole, and the first sealing element is received in the first adapter unit such that a proximally protruding portion of the first sealing element extends through the first through-hole of the proximal wall and is formed to protrude in the proximal direction relative to the proximal wall of the first adapter unit; the cylindrical component of the second adapter unit has a distal wall having a through-hole, and the second sealing element is received in the cylindrical component of the second adapter unit such that a distally protruding portion of the second sealing element extends through the through-hole of the distal wall and is formed to protrude in the distal direction relative to the distal wall of the cylindrical component; and the proximal wall of the first adapter unit and the distal wall of the cylindrical component of the second adapter unit are brought into abutment/the proximal wall is held close to the distal wall when the first adapter unit and the second adapter unit are transferred from the unconnected or separated state (via/passing the transfer state) to the connected state.

The sealing elements are preferably made of an elastic material, in particular an elastomer.

When the sealing elements each have portions protruding from a wall, the preferably resilient sealing elements are resiliently/flexibly pressed together to exert the predetermined/defined/constant pressure on each other when the proximal wall of the first adapter unit is held close to/comes into abutment with the distal wall of the cylindrical component. According to the present disclosure, the proximal wall and the distal wall are in abutment when they are in contact with each other or when they are arranged very close to each other/minimally spaced apart. For example, the proximal wall and the distal wall may be in contact/touch each other in the connected state of the two adapter units or may still be minimally spaced apart as they are pushed away from each other by the two compressed sealing elements being in contact/in abutment with each other.

If the adapter units each have distal/proximal end walls and the sealing elements protrude with respect to the end walls, good accessibility and thus good disinfectability of the two adapter units is advantageously provided.

In other words, it can be said that the two adapter units as a whole are preferably substantially cylindrical/rotationally symmetrical and are convex (and thus not concave) at their axial ends, which are to be connected to each other in order to establish the connected state of the two adapter units, i.e. they preferably have portions/parts projecting only in the axial direction, in particular away from the distal wall of the second adapter unit (in the distal direction) and away from the proximal wall of the first adapter unit (in the proximal direction). In particular, both adapter units are convex in their unconnected/separated state to allow good accessibility and disinfectability in the separated state of the adapter units.

Preferably, the cylindrical component of the second adapter unit comprises an engagement element which is transferred from a non-engaged state to an engaged state by the hollow-cylindrical/sleeve-shaped component during the translational movement of the hollow-cylindrical/sleeve-shaped component relative to the cylindrical component caused by the rotation/turning of the hollow-cylindrical/sleeve-shaped component.

Particularly preferably, at least two engagement elements are provided. The two engagement elements may be diametrically opposite each other. More than two engagement elements, for example three, four, five, six, etc., may also be provided.

The engagement element is preferably configured as an elastic/spring arm/snap arm. In the unconnected/separated state, the engagement element can be prestressed into a non-engaged state, for example radially outward. In particular, the engagement element can be received in the non-engaged state in a receiving recess in the sleeve-shaped/hollow-cylindrical component of the second adapter unit. The engagement element/arm preferably comprises a latching hook at its distal end. Preferably, portions of the arm, in particular the latching hook, protrude with respect to the distal wall of the cylindrical component, i.e. the latching hook is preferably arranged distally protruding with respect to the distal wall.

Advantageously, the first adapter unit comprises an engagement element receptacle (e.g. a flange/flange portion) for the engagement element and the engagement element is received in the engagement element receptacle in the engagement state, wherein in the engagement state, the engagement element holds the proximal wall of the first adapter unit in contact with/near the distal wall of the cylindrical component and thus generates a counterforce against the pushing-apart force of the two sealing elements. In particular, the first adapter unit and the second adapter unit are in the transfer state as soon as the engagement element is received in the engagement element receptacle.

In other words, the first sealing element and the second sealing element are elastically pressed together/compressed/clinched when a user presses the first adapter unit against the second adapter unit (pressing movement). The first sealing element and the second sealing element preferably generate a pushing-apart force to return to their uncompressed, original initial state. When the engagement element is received in the engagement element receptacle, the first adapter unit and the second adapter unit are preferably held together and the first sealing element and the second sealing element are prevented from (fully) releasing/relaxing and returning to their original initial state. Thus, it is achieved that the first and the second sealing element exert a constant, defined and predetermined force or a constant, defined and predetermined pressure on each other in the transfer state.

The first adapter unit preferably has a flange portion, in particular at its proximal end. The flange portion preferably protrudes radially outward. The flange portion serves in particular as an engagement element receptacle for the engagement element (in particular the latching hook of the engagement element) of the cylindrical component of the second adapter unit.

According to a preferred embodiment, the first adapter unit may have at its proximal end a circumferential flange portion protruding radially outward with at least two, preferably diametrically opposed, recesses/cut-outs/material cut-outs (for the engagement element of the cylindrical component of the second adapter unit, in particular for the latching hook). More than two, for example three, four, five or six recesses may also be provided, in which case the plurality of recesses is preferably evenly distributed in circumferential direction. Two recesses may also be diametrically opposite each other.

It is advantageous if the first adapter unit has a first port, in particular a Luer lock port, and/or the second adapter unit has a second port, in particular a Luer lock port. For example, the first adapter unit may have the first port at an axial end, in particular at its distal end. The first port may be a Luer lock port, in particular a female Luer lock port. The second adapter unit may have the second port at an axial end, particularly at its proximal end. The second port may be formed as a Luer lock port, particularly a male Luer lock port. The ports may also be adapted to a container, for example an ampule, to which the corresponding adapter unit is to be connected, i.e. the ports do not necessarily have to be configured as Luer lock ports.

The first adapter unit preferably has an internal fluid passage extending in an axial direction of the first adapter unit. The first adapter unit may have a substantially hollow-cylindrical component containing the proximal wall. Advantageously, the sealing element disposed at the proximal end of the first adapter unit/of the hollow-cylindrical component comprises a first cylindrical portion and a second cylindrical portion, wherein a diameter of the first cylindrical portion is greater than a diameter of the second cylindrical portion, and wherein the second cylindrical portion of the first sealing element extends through the through-hole of the proximal wall of the hollow-cylindrical component and protrudes in the proximal direction. The first adapter unit may further comprise a first port part inserted into and connected to/fixed to the hollow-cylindrical component, the first port part having the first port at its distal end, and the first port part having/forming the inner fluid passage. The inner fluid passage may be hollow-cylindrical in shape. Preferably, a proximal end of the inner fluid passage presses the first sealing element against the proximal wall of the hollow-cylindrical component. The flange portion protruding radially outward is preferably provided on the hollow-cylindrical component of the first adapter unit.

The second sealing element, which is preferably received in the cylindrical component of the second adapter unit, may comprise a first cylindrical portion and a second cylindrical portion, wherein a diameter of the first cylindrical portion is larger than a diameter of the second cylindrical portion, and wherein the second cylindrical portion of the second sealing element extends through the through-hole of the distal wall of the cylindrical component and protrudes in the distal direction.

In addition to the hollow-cylindrical/sleeve-shaped component and the cylindrical component, the second adapter unit preferably also has a second port part, in particular at its proximal end. The second port part is preferably firmly connected to the hollow-cylindrical/sleeve-shaped component. The second port part preferably comprises the second port. Furthermore, the second port part preferably has a needle receptacle, to/in which the needle is fixedly attached.

According to a preferred embodiment, the second sealing element is rather large. According to an alternatively preferred embodiment, in order to reduce friction between the second sealing element and the needle, which is located in the second sealing element in the unconnected state and in the transfer state of the two adapter units, the second adapter unit may also comprise a third sealing element and a spacer element arranged between the second sealing element and the third sealing element. According to this alternative embodiment, the second sealing element and the third sealing element may be rather small (i.e. with a smaller axial extension) and an annular/sleeve-shaped spacer element may be provided between the second sealing element and the third sealing element, within which the needle can be moved without friction.

Another preferred embodiment is characterized in that the cylindrical component comprises a guide element, and the first adapter unit comprises a guide-element receptacle for preventing relative twisting between the cylindrical component and the first adapter unit during rotation/turning of the hollow-cylindrical/sleeve-shaped component. The pressing and rotation movement to be performed by the user is advantageously supported by the guide element and the guide-element receptacle.

For example, the distal wall of the cylindrical component of the second adapter unit may have one or more projections (e.g., two) that serve as the guide element. The projections may, for example, be substantially arcuate. The guide element receptacle of the first adapter unit may be realized as a recess, for example as a recess in a flange portion of the first adapter unit.

In addition, the present disclosure relates to a use of a fluid transfer device as described above for transferring or conveying medicinal products, in particular CMR medicinal products, from a first container to a second container.

Furthermore, the present disclosure relates to a closed medicine transfer system comprising a fluid transfer device as described above and at least a first container, wherein the fluid transfer device is adapted to provide a fluid connection between the first container and a second container or between the first container and a patient.

The first container may be an ampule, a syringe, an intravenous (IV) infusion bag, etc. For example, the first container is an ampule and the second container is a syringe, or the first container is a syringe and the second container is an intravenous infusion bag. Preferably, when the first container is the infusion bag, a fluid transfer occurs from the infusion bag to a patient.

The fluid transfer device, which may also be referred to as a port system/coupling system, and the closed medicine transfer system of the present disclosure suitably prevent transfer of impurities/contaminants from the environment into the system/device and escape of hazardous/harmful drug or vapor concentrations out of the system/device (mechanically). In other words, the fluid transfer device and the closed medicine transfer system serve a contamination-free transfer/conveying of fluids. By applying a constant, defined, predetermined pressure to the first sealing element and the second sealing element before the fluid connection is made, it is achieved that no/hardly any residual moisture remains on the surfaces of the two sealing elements, so that dry connection and separation is made possible. Due to the convex configuration of the corresponding connection ends of the two adapter units, good accessibility of the coupling surfaces and thus good disinfectability is ensured. The fluid transfer device of the present disclosure is also easy to handle and compact.

### BRIEF DESCRIPTION OF THE DRAWINGS

The disclosure is further explained below with the aid of Figures. The following is shown:

FIG. 1 shows a perspective view of a fluid transfer device with two adapter units, wherein the fluid transfer device is integrated in a medicine transfer system;

FIG. 2 shows a side view of the fluid transfer device in a separated state of the two adapter units;

FIG. 3 shows a sectional view of the fluid transfer device in the separated state of the two adapter units;

FIG. 4 shows a side view of the fluid transfer device in a transfer state of the two adapter units;

FIG. 5 shows a sectional view of the fluid transfer device in the transfer state of the two adapter units;

FIG. 6 shows a side view of the fluid transfer device in a connected state of the two adapter units;

FIG. 7 shows a sectional view of the fluid transfer device in the connected state of the two adapter units;

FIG. 8 shows a sectional view of the fluid transfer device in a separated state of the two adapter units according to a first alternative embodiment; and

FIG. 9 shows a perspective view of the fluid transfer device in a separated state of the two adapter units according to a second alternative embodiment.

### DETAILED DESCRIPTION

The Figures are merely schematic in nature and are intended solely for the purpose of understanding the present disclosure. Identical elements are provided with the same reference signs. The features of the individual embodiments/examples can be interchanged.

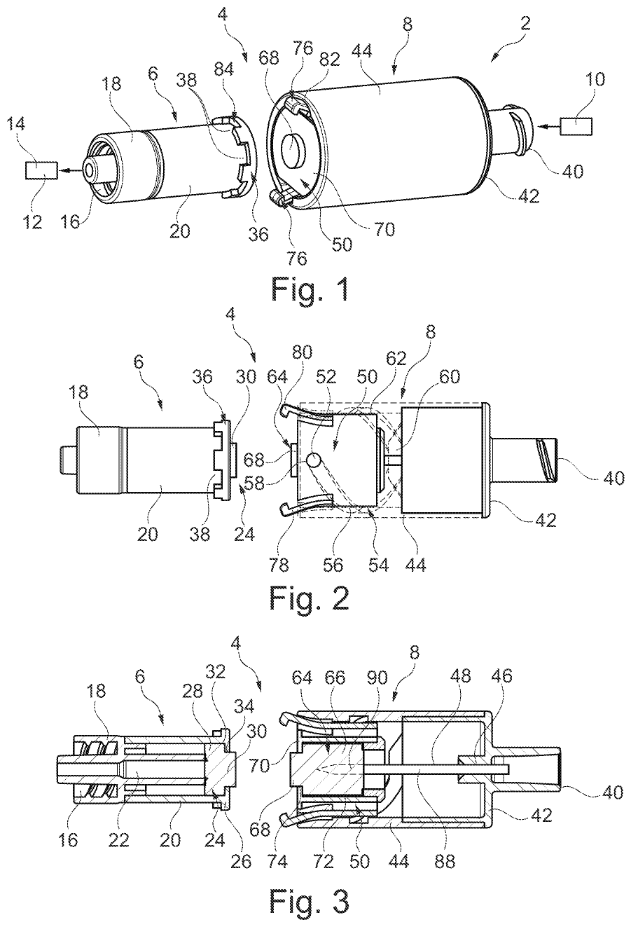

FIG. 1 shows a closed medicine transfer system 2. The closed medicine transfer system 2 comprises a fluid transfer device 4 with a first adapter unit 6 and a second adapter unit 8. The first adapter unit 6 and the second adapter unit 8 are shown in FIG. 1 in an unconnected or separated state. The first adapter unit 6 and the second adapter unit 8 can in principle be brought into a connected state, in which a fluid connection is provided between the first adapter unit 6 and the second adapter unit 8. In the connected state of the first adapter unit 6 and the second adapter unit 8, hazardous/harmful medicinal products, such as CMR medicinal product, can be transferred from a first container 10 to a second container 12 or a patient 14 via the fluid transfer device 4 without contamination. The fluid transfer device 4 is thus basically configured to establish a fluid connection between the first container 10 and the second container 12/the patient 14. For example, the first container 10 is an ampule, and a fluid from the ampule can be transferred via the fluid transfer device 4 to a second container 12 configured as a syringe. Alternatively, the first container 10 may be a syringe, and a fluid may be transferred from the syringe via the fluid transfer device 4 to a second container 12 configured as an infusion bag. Further alternatively, the first container 10 may be an infusion bag, and a fluid may be transferred from the infusion bag to the patient 14 via the fluid transfer device 4.

FIG. 1, FIG. 2 and FIG. 3 show the fluid transfer device 4 in the unconnected/separated state of the two adapter units 6, 8. The first adapter unit 6 and the second adapter unit 8 are each substantially cylindrical. For the following explanations, it is assumed that the second adapter unit 8 is located closer to a medical staff/user and is thus arranged proximally with respect to the first adapter unit 6 and the first adapter unit 6 is arranged distally with respect to the second adapter unit 8.

The first adapter unit 6 has a first port 16 at its distal end, which is configured as a female Luer lock port. The first port 16 is part of a first port part 18, which in the assembled state of the first adapter unit 6 is firmly connected to a (first) substantially hollow-cylindrical component 20. In particular, it can be seen from FIG. 3 that the port part 18 is formed in the manner of a plug-in socket and is simply plugged into the hollow-cylindrical component 20. The port part 18 has an internal fluid passage 22 that extends through the first adapter unit 6/in an axial direction of the first adapter unit 6 to a first sealing element 24. When the port part 18 and the hollow-cylindrical component 20 are connected to each other, the inner fluid passage 22 presses the first sealing element 24 against a proximal wall 26 of the hollow-cylindrical component 20.

The first sealing element 24 is an elastic sealing element and is made of an elastomer. The first sealing element 24 includes a first cylindrical portion 28 and a second cylindrical portion 30. A diameter of the first cylindrical portion 28 is greater than a diameter of the second cylindrical portion 30. The first cylindrical portion 28 abuts the proximal wall 26 and an inner lateral surface 32 of the hollow-cylindrical component 20 from the inside. The second cylindrical portion 30 extends through a first through-hole 32 provided in the proximal wall 26 and protrudes with respect to the proximal wall 26 in the proximal direction.

The hollow-cylindrical component 20 of the first adapter unit 6 has a circumferential flange portion 36 protruding radially outward at its proximal end. The flange portion 36 has a plurality of (in this case six) recesses/cut-outs/material cut-outs 38. The recesses 38 are evenly distributed in the circumferential direction and two recesses 38 are diametrically opposite each other.

The second adapter unit 8 has a second port 40 at its proximal end, which is configured here as a male Luer lock port. The second port 40 is part of a second port part 42, which in the assembled state of the second adapter unit 8 is firmly connected to a (second) substantially hollow-cylindrical component 44. In particular, it can be seen from FIG. 3 that the port part 42 is formed in the manner of a plug-in socket and is simply plugged into the hollow-cylindrical component 44. The port part 42 has a needle receptacle 46, in which a hollow needle 48 is firmly held/fixed.

The second adapter unit 8 further comprises a substantially cylindrical component 50 received at a distal end of the second adapter unit 8 in the hollow-cylindrical component 44. In particular, as shown in FIG. 2, the cylindrical component 50 includes a pin-shaped projection 52 projecting radially outward (from a lateral surface). The projection 52 is received in a motion link/motion-link path 54 of the hollow-cylindrical component 44. The motion link/motion-link path 54 is formed as a groove-like recess and substantially comprises a first portion 56, in which the motion link 54 extends from a distal stop 58 in a thread-like manner/in both the circumferential direction and the axial direction (distal-proximal), i.e. obliquely, a second portion 60, which is a portion without inclination, that is, a portion in which the motion link 54 extends only in the circumferential direction, and a third portion 62 which extends from the second portion 60 in a thread-like manner/in both the circumferential direction and the axial direction (proximal-distal), that is, obliquely, to a second distal stop (not shown).

The cylindrical component 50 of the second adapter unit 8 has a second sealing element 64 on the inside. The second sealing element 64 is an elastic sealing element and is made of an elastomer. The second sealing element 64 extends in the axial direction over approximately the entire length of the cylindrical component 50. The second sealing element 64 comprises a first cylindrical portion 66 and a second cylindrical portion 68. A diameter of the first cylindrical portion 66 is greater than a diameter of the second cylindrical portion 68. The first cylindrical portion 66 abuts a distal wall 70 and an inner lateral surface 72 of the cylindrical component 50 from the inside. The second cylindrical portion 68 extends through a second through-hole 74 provided in the distal wall 70 and protrudes in the distal direction with respect to the distal wall 70.

The cylindrical component 50 of the second adapter unit 8 has two diametrically opposed engagement elements 76, each having an elastically resilient arm/snap arm 78 and a latching hook 80. In the separated state of the two adapter units 6, 8 shown in FIGS. 1 to 3, the engagement elements 76 project distally with respect to a distal end of the hollow-cylindrical component 44 and with respect to the distal wall 70 of the cylindrical component 50. In particular, the distal wall 70 is disposed approximately level with the distal end of the hollow-cylindrical component 44 in the axial direction or may also project somewhat in the distal direction with respect to the distal end of the hollow-cylindrical component 44. The engagement elements 76 are prestressed radially outward (in a non-engagement state) and are each received in a receiving recess 82 provided in the hollow-cylindrical component 44. The receiving recesses 82 are groove-like/trough-like/channel-like recesses in the hollow-cylindrical component 44.

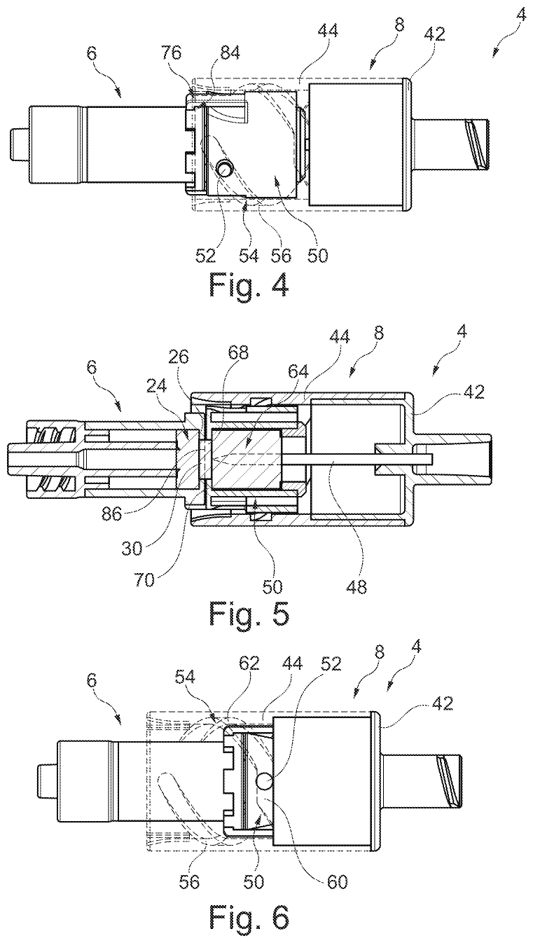

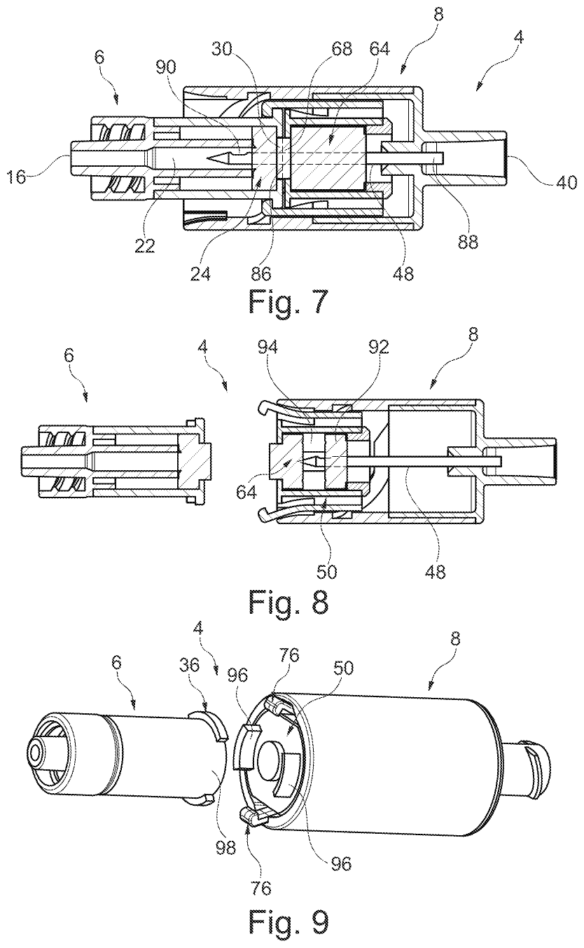

FIG. 4 and FIG. 5 each show a transfer state of the two adapter units 6, 8. FIG. 6 and FIG. 7 each show a connected state of the two adapter units 6, 8. According to the present disclosure, when the two adapter units 6, 8 are connected to each other, they first transition from the unconnected/separated state (see FIGS. 1 to 3) to the transfer state (see FIGS. 4 and 5) and only then from the transfer state to the connected state (see FIGS. 6 and 7).

In order to transfer the two adapter units 6, 8 from their separated state to the transfer state, a user/medical personnel presses the two adapter units 6, 8 against/toward each other (translationally or in the axial direction of the two adapter units 6, 8), so that the first sealing element 24, in particular the protruding second cylindrical portion 30 of the first sealing element 24, is pressed against the second sealing element 64, in particular the protruding second cylindrical portion 68 of the second sealing element 64. The distal wall 70 of the cylindrical component 50 of the second adapter unit 8 is now arranged in contact with or at least very close to the proximal wall 26 of the hollow-cylindrical component 20 of the first adapter unit 6. The user/medical staff preferably orientates the two adapter units 6, 8 in such a way that the engagement elements 76 of the second adapter unit 8 are arranged in the same position in the circumferential direction as the recesses 38 of the first adapter unit 6. The recesses 38 serve in particular as engagement element receptacles 84 and are preferably adapted in their shape and size to the engagement elements 76, in particular to the latching hooks 80 of the engagement elements 76.

When the two adapter units 6, 8 are pressed onto/against each other, the user/medical staff performs a rotational movement of the two adapter units 6, 8 relative to each other. In particular, the user turns the hollow-cylindrical component 44 (and the second port part 42, which is firmly connected to the hollow-cylindrical component 44) clockwise. Due to the pressure which the user applies from the first adapter unit 6 to the cylindrical component 50 of the second adapter unit 8, the first adapter unit 6 and the cylindrical component 50 of the second adapter unit preferably remain at rest, i.e. they do not rotate.

As can be seen in particular from a comparison of FIG. 2 with FIG. 4, the pin-shaped projection 52 of the cylindrical component 50 now travels in the motion link/motion-link path 54 of the hollow-cylindrical component 44. The traveling of the projection 52 in the motion link 54 simultaneously causes the hollow-cylindrical component 44 to slide in a translational direction (in a distal direction) over the engagement elements 76 of the cylindrical component 50, pushing them radially inwards against their spring pretension. In other words, the latching hooks 80 of the engagement elements 76 are received in the recesses 38 of the flange portion 36 (flanked by projections of the flange portion 36).

In this state (transfer state), the first adapter unit 6 can no longer be moved/separated from the cylindrical component 50 of the second adapter unit 8 and can also no longer be turned relative to the cylindrical component 50 of the second adapter unit 8. The first adapter unit 6 and the cylindrical component 50 of the second adapter unit 8 are thus attached/connected to each other in a defined manner. In this state, the first sealing element 24 (in particular its protruding second cylindrical portion 30) and the second sealing element 64 (in particular its protruding second cylindrical portion 68) are elastically compressed/clinched. The two sealing elements 24 and 64 therefore exert a force on/against each other, which pushes the first adapter unit 6 away from the second adapter unit 8. In the transfer state, the engagement elements 76 are received in the engagement element receptacles 78 and exert a counterforce that prevents the two adapter units 6, 8 from separating from each other. The two sealing elements 24, 64 can thus no longer reach their relaxed initial state in the transfer state and thus exert a constant, defined, predetermined force or a constant, defined, predetermined pressure on each other.

It is self-evident for the skilled person that he can adjust the constant, defined, predetermined pressure appropriately by suitable dimensioning of the two sealing elements 24, 64 (elasticity, protruding portions), the engagement elements 76 and the engagement element receptacles 84.

The two adapter units 6, 8 preferably reach the transfer state after a predetermined turning around a first predetermined angle α. As can be seen in particular from FIG. 5, the hollow needle 48, which moves together with the hollow-cylindrical component 44 and the second port part 42, is still stuck in the second sealing element 64 in the transfer state. That is, the hollow needle 48 has not yet penetrated an interface 86 between the first sealing element 24 and the second sealing element 64 in the transfer state. Thus, according to the present disclosure, the predetermined, constant, defined pressure at the interface 86 between the first sealing element 24 and the second sealing element 64 is first established before the interface 86 is penetrated by the hollow needle 48.

If the hollow-cylindrical component 44 is now turned further clockwise by a second predetermined angle R from the transfer state, the pin-shaped projection 52 of the cylindrical component 50 travels further along the first portion 56 of the motion link 54. As a result, the hollow-cylindrical component 44 (and thus also the hollow needle 48) is moved further in a translational manner (in the distal direction). The hollow needle 48 (pencil point needle) thereby completely penetrates the interface 86 between the first sealing element 24 and the second sealing element 64 and also the first sealing element 24 of the first adapter unit 6.

When the user notices during the turning of the hollow-cylindrical component 44 that the hollow-cylindrical component 44 no longer moves translationally (in the distal direction) with respect to the first adapter unit 6 (this is the case starting from the separated state approximately after a 180° turn), the connected state of the two adapter units 6, 8 is reached. In the connected state, the pin-shaped projection 52 is located in the second portion 60 of the motion link 54, which extends only in the circumferential direction.

In the connected state of the two adapter units 6, 8 shown in FIG. 7, a fluid connection is now provided between the two adapter units 6, 8. In particular, a fluid, for example a CMR medicinal product, can flow into an inner fluid passage 88 of the hollow needle 48 via the second port 40, flow out of the inner fluid passage 88 of the hollow needle 48 via a lateral opening 90 of the hollow needle 48, and leave the first adapter unit 6 and thus the entire fluid transfer device 4 via the inner fluid passage 22 of the first adapter unit 6 and the port 16. Alternatively, the fluid can also flow into the inner fluid passage 22 of the first adapter unit 6 via the port 16 of the first adapter unit 6, enter the inner fluid passage 88 of the hollow needle 48 via the lateral opening 90 of the hollow needle 48, and leave the second adapter unit 8 and thus the entire fluid transfer device 4 again via the second port 40.

When the fluid (CMR medicinal product) has been completely transferred from the first container 10 to the second container 12/the patient 14, the fluid transfer device 4 can be moved again from the connected state of the two adapter units 6, 8 via the transfer state to the unconnected/separated state. Starting from the connected state of the two adapter units 6, 8, the user can perform a clockwise or counterclockwise rotation/turning. In a counterclockwise rotation, the pin-shaped projection 52 moves from the second portion 60 of the motion link 54 back into the first portion 56 of the motion link 54 and thus back to the first distal stop 58. In a clockwise rotation, the pin-shaped projection 52 travels from the second portion 60 of the motion link 54 into the third portion 62 of the motion link 54 and towards the (not shown) second distal stop.

When the two adapter units 6, 8 change from the connected state to the transfer state, the first sealing element 24 and the second sealing element 64 apply the defined/constant/predetermined pressure against each other until the hollow needle 48 has been retracted again via the interface 86 of the first sealing element 24 and the second sealing element 64. The mechanical separation of the two adapter units thus only takes place when the hollow needle 48 has already been completely retracted again. Thus, the fluidically connected state and the fluidically unconnected state of the fluid transfer device 4 are only established when the constant/defined/predetermined pressure is applied to the two sealing elements 24, 64.

The first sealing element 24 and the second sealing element 64 are each reclosable/self-sealing sealing elements. That is, when the hollow needle 48 is retracted, the first sealing element 24 seals the first through-hole 34 and the second sealing element 64 seals the second through-hole 74. Thus, fluid transfer out of the first adapter unit 6 and the second adapter unit 8 is prevented in the unconnected state/separated state.

In the embodiment example shown in FIGS. 1 to 7, the second sealing element 64 is very large or voluminous. This is associated with relatively high friction between the second sealing element 64 and the hollow needle 48. FIG. 8 shows an alternative embodiment according to which the second sealing element 64 is smaller, in particular with a smaller axial extension. In the embodiment example shown in FIG. 8, a third sealing element 92 and an annular/sleeve-shaped spacer element 94 are provided. The second sealing element 64, the spacer element 94 and the third sealing element 92 are received (in this order) in the cylindrical component 50 and are in contact with each other (the spacer element 94 is located between the two sealing elements 64, 92).

In the embodiment shown in FIGS. 1 to 7, the relative positioning of the two adapter units 6, 8 when pressed against/on top of each other may be somewhat difficult, since the engagement elements 76 must be in the same position in circumferential direction as the recesses 38 of the flange portion 36. FIG. 9 shows an alternative embodiment according to which the cylindrical component 50 has two guide elements 96 in the form of circular arc projections and the first adapter unit 6 has two guide-element receptacles 98 in the form of recesses in the flange portion 36. According to this embodiment, the flange portion 36 is not circumferential but is interrupted in circumferential direction for the guide-element receptacles 98. The circular arc projections are shaped and sized to match the interruptions in the flange portion 36. When the guide elements 96 are received in the guide-element receptacles 98, the first adapter unit 6 can no longer rotate with respect to the cylindrical component 50 of the second adapter unit 8. The engagement elements 76 engage directly with the flange portion 36 (i.e., no recesses 38 are required in the flange portion 36). Thus, the flange portion 36 itself forms the engagement element receptacle 84.

1. A fluid transfer device for use in medical and/or pharmacological applications, comprising a first adapter unit comprising a first sealing element and a second adapter unit comprising a second sealing element, wherein the first adapter unit and the second adapter unit are configured to provide a fluid connection between the first adapter unit and the second adapter unit in a connected state and to prevent a fluid transfer out of the first adapter unit and the second adapter unit in an unconnected or separated state, wherein
the first adapter unit and the second adapter unit are formed to establish the fluid connection between the first adapter unit and the second adapter unit by a combined pressing movement toward each other and rotational movement with respect to each other only after adjusting a constant or defined pressure, which the first sealing element and the second sealing element apply against each other, wherein
the first adapter unit and the second adapter unit are configured to assume, in addition to the connected state and the unconnected or separated state, a transfer state in which the first adapter unit and the second adapter unit are attached to each other and the first sealing element and the second sealing element are in contact with each other and apply the constant or defined pressure against each other, and a fluid connection is not yet provided between the first adapter unit and the second adapter unit, and wherein
the first adapter unit and the second adapter unit are configured to be transferred from the unconnected or separated state to the transfer state by pressing on each other and rotating relative to each other by a first predetermined angle, and to be transferred from the transfer state to the connected state by continued rotation by a second predetermined angle.

2. The fluid transfer device according to claim 1, wherein the second adapter unit comprises a hollow needle forming a fluid passage, and the hollow needle is received in the second sealing element in the unconnected state or the transfer state and penetrates an interface between the first sealing element and the second sealing element only when the first adapter unit and the second adapter unit are transferred from the transfer state to the connected state.

3. The fluid transfer device according to claim 1, wherein the second adapter unit comprises a substantially hollow-cylindrical component and a substantially cylindrical component, wherein the cylindrical component is received in the hollow-cylindrical component, and wherein rotation of the hollow-cylindrical component causes translational movement of the hollow-cylindrical component and the cylindrical component relative to each other.

4. The fluid transfer device according to claim 3, wherein the cylindrical component has a pin-shaped projection projecting radially outward, and the hollow-cylindrical component has a motion link formed like a thread, and the pin-shaped projection of the cylindrical component is received in the motion link of the hollow-cylindrical component.

5. The fluid transfer device according to claim 3, wherein
the first adapter unit has a proximal wall having a first through-hole, and the first sealing element is received in the first adapter unit such that a proximally protruding portion of the first sealing element extends through the first through-hole of the proximal wall and is formed to protrude in the proximal direction relative to the proximal wall of the first adapter unit,
the cylindrical component of the second adapter unit has a distal wall having a through-hole, and the second sealing element is received in the cylindrical component of the second adapter unit such that a distally protruding portion of the second sealing element extends through the through-hole of the distal wall and is formed to protrude in the distal direction relative to the distal wall of the cylindrical component, and
the first sealing element and the second sealing element are brought into contact when the first adapter unit and the second adapter unit are transferred from the unconnected or separated state into the connected state.

6. The fluid transfer device according to claim 3, wherein the cylindrical component of the second adapter unit comprises an engagement element which is transferred from a non-engaged state to an engaged state by the hollow-cylindrical component during the translational movement of the hollow-cylindrical component relative to the cylindrical component caused by the rotation of the hollow-cylindrical component.

7. The fluid transfer device according to claim 6, wherein the first adapter unit comprises an engagement element receptacle for the engagement element and the engagement element is received in the engagement element receptacle in the engagement state, wherein in the engagement state, the engagement element holds the first sealing element in contact with the second sealing element and generates a counterforce against a pushing-apart force of the two sealing elements.

8. The fluid transfer device according to claim 3, wherein the second adapter unit comprises a third sealing element and a spacer element arranged between the second sealing element and the third sealing element.

9. The fluid transfer device according to claim 3, wherein the cylindrical component comprises a guide element, and the first adapter unit comprises a guide-element receptacle for preventing relative twisting between the cylindrical component and the first adapter unit during rotation of the hollow-cylindrical component.

10. The fluid transfer device according claim 1, wherein the sealing elements are made of an elastic material, in particular an elastomer.

11. The fluid transfer device according to claim 1, wherein the first adapter unit has a first port, and the second adapter unit has a second port.

12. A method of transferring a medicinal product contained in a first container to a second container, the method comprising the steps of:
attaching the fluid transfer device according to claim 1 to the first container and the second container; and
transferring the medicinal product from the first container to the second container.

13. A closed medicine transfer system comprising:
a fluid transfer device according to claim 1; and
at least a first container,
wherein the fluid transfer device is configured to provide a fluid connection between the first container and a second container or between the first container and a patient.
